(19) United States
(12) Patent Application Publication
(10) Pub. No.: US 2018/0104516 A1
(43) Pub. Date: Apr. 19, 2018

(54) NOSE PAD FOR MEDICINAL APPLICATION

(71) Applicant: Aromeyes, LLC, Monmouth Beach, NJ (US)

(72) Inventors: Robert Fegan, Monmouth Beach, NJ (US); Evan Krawitz, Morganville, NJ (US)

(21) Appl. No.: 15/724,485

(22) Filed: Oct. 4, 2017

Related U.S. Application Data
(60) Provisional application No. 62/407,782, filed on Oct. 13, 2016.

(51) Int. Cl. A62B 9/06; G02C 5/12

(52) U.S. Cl. CPC A62B 9/06; A61M 21/02; A61M 2210/0618; G02C 5/12

(57) ABSTRACT

Disclosed herein are aromatherapy delivery systems, methods of their use and preparation as well as aromatherapy delivery system kits. The aromatherapy delivery systems depicted herein are suitable for medicinal applications, such as the treatment of common human ailments.

### FIELD OF THE INVENTION

The present invention relates to an aromatherapy delivery system, methods of using the aromatherapy delivery system, methods of preparing the aromatherapy delivery system, and kits containing the aromatherapy delivery system. More particularly, to an aromatherapy delivery system for medicinal applications.

### BACKGROUND OF THE INVENTION

Aromatherapy is the practice of using medicinal agents such as natural oils extracted from flowers, bark, stems, leaves, roots or other parts of a plant to enhance psychological and physical well-being. The inhaled aroma from these oils is widely believed to stimulate brain function and provide homeopathic and/or allopathic remedies to common human ailments.

These oils can be directly inhaled through the nose, delivered through the eyes via aerial diffusion, and absorbed through the skin topically to be subsequently transferred into the blood stream and promote healing of the whole body.

It is a form of alternative medicine that is gaining momentum and is being offered to supplement standard treatments and/or to replace certain treatments. It is offered for a variety of applications including pain relief, mood and energy enhancement, anxiety reduction, relaxation, and increased cognitive function to name a few.

Currently, various systems are being employed to deliver aromatherapy. Some systems deliver aroma to the immediate environment such as aroma lamps or diffusers, while other systems are facial aromatherapy delivery systems such as eye masks and nose plugs. These systems are cumbersome and not very discreet making the delivery of such aromatherapy inconvenient and less likely to be continuous.

There is a need for an improved facial aromatherapy delivery system that will be able to deliver discreetly and continuously individualized medicinal products that may assist in treating common human ailments.

### SUMMARY

Broadly, an embodiment of the present invention provides an aromatherapy delivery system comprising: a cover configured to receive a nose support device through an opening. The cover may comprise an inner section. The inner section may be configured to receive a medicinal product.

Another embodiment of the present invention provides an aromatherapy delivery system comprising: a pair of eyeglasses and a cover. The pair of eyeglasses may comprise a right eye frame, a left eye frame, a bridge connecting between the right eye frame and the left eye frame, and a nose support device that may be attached to the right eye frame, the left eye frame, the bridge, or a combination thereof. The cover may be configured to receive a nose support device through an opening. The cover may comprise an inner section. The inner section may be configured to receive a medicinal product.

Certain embodiments of the present invention are directed towards a method for treating an ailment by emitting aromatherapy to an individual's nose or eyes through an aromatherapy delivery system disclosed herein. For instance the aromatherapy delivery system may comprise a cover configured to receive a nose support device through an opening. The cover may comprise an inner section. The inner section may be configured to receive a medicinal product.

Certain other embodiments of the present invention are directed towards methods of forming an aromatherapy delivery system. In some embodiments, the method comprises slipping a cover over a nose support device. The cover may be configured to receive a nose support device through an opening. The cover may comprise an inner section and the inner section may be configured to receive a medicinal product.

In other embodiments, the method comprises lining a cover for a nose support device with a medicinal product. The cover may be configured to receive a nose support device through an opening. The cover may comprise an inner section and the inner section may be configured to receive a medicinal product.

Yet other embodiments of the invention are directed towards a kit. The kit may comprise a pair of eyeglasses and a cover. The pair of eyeglasses may comprise a right eye frame, a left eye frame, a bridge connecting between the right eye frame and the left eye frame, and a nose support device that may be attached to the right eye frame, the left eye frame, the bridge, or a combination thereof. The cover may be configured to receive a nose support device through an opening. The cover may comprise an inner section. The inner section may be configured to receive a medicinal product.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1 is a perspective view of a pair of eyeglasses according to an embodiment;

FIGS. 2A and 2B are blown up perspective views of a nose pad according to an embodiment;

FIG. 3A is a perspective view of a cover for a nose support device according to an embodiment;

FIG. 3B depicts a cover for a nose support device according to an embodiment;

FIG. 4 is a demonstration of the flexibility of a cover for a nose support device according to an embodiment;

FIG. 5 depicts a side view of a cover for a nose support device with medicinal product without a nose support device;

FIG. 6 depicts a top view of a cover for a nose support device with medicinal product without a nose support device;

FIG. 7 depicts a side view of a cover for a nose support device with medicinal product coated over a nose support device;

FIG. 8 depicts a top view of a cover for a nose support device with medicinal product coated over a nose support device;

FIG. 9 depicts slipping a cover over a nose support device.

### DETAILED DESCRIPTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, the present disclosure is directed to an aromatherapy delivery system that could comprise a cover comprising an inner section such that the inner section may be configured to receive a medicinal product and the cover may be configured to receive a nose support device through an opening. The aromatherapy delivery system may further comprise a pair of eyeglasses. In some embodiments, the cover may be already attached to the eyeglasses. In other embodiments, the cover may be provided separately from the eyeglasses in a kit. If the cover is provided separately from the eyeglasses, an aromatherapy delivery system may be formed by slipping the cover over a nose support device in the eyeglasses. In some embodiments, the cover may include a medicinal product. In other embodiments, the aromatherapy delivery system may be formed by lining the cover with a medicinal product. The aromatherapy delivery systems disclosed herein may be used to treat various ailments by emitting aromatherapy to an individual's eyes and/or nose.

The aromatherapy delivery systems disclosed herein are more cosmetically discreet mobile delivery systems than eye masks and nose pads that are presently used. The present invention delivers continuous aromatherapy with close proximity to an individual's eyes and nose. Thereby, making the present invention beneficial as a homeopathic and/or allopathic remedy for alleviating ocular and other common human ailments.

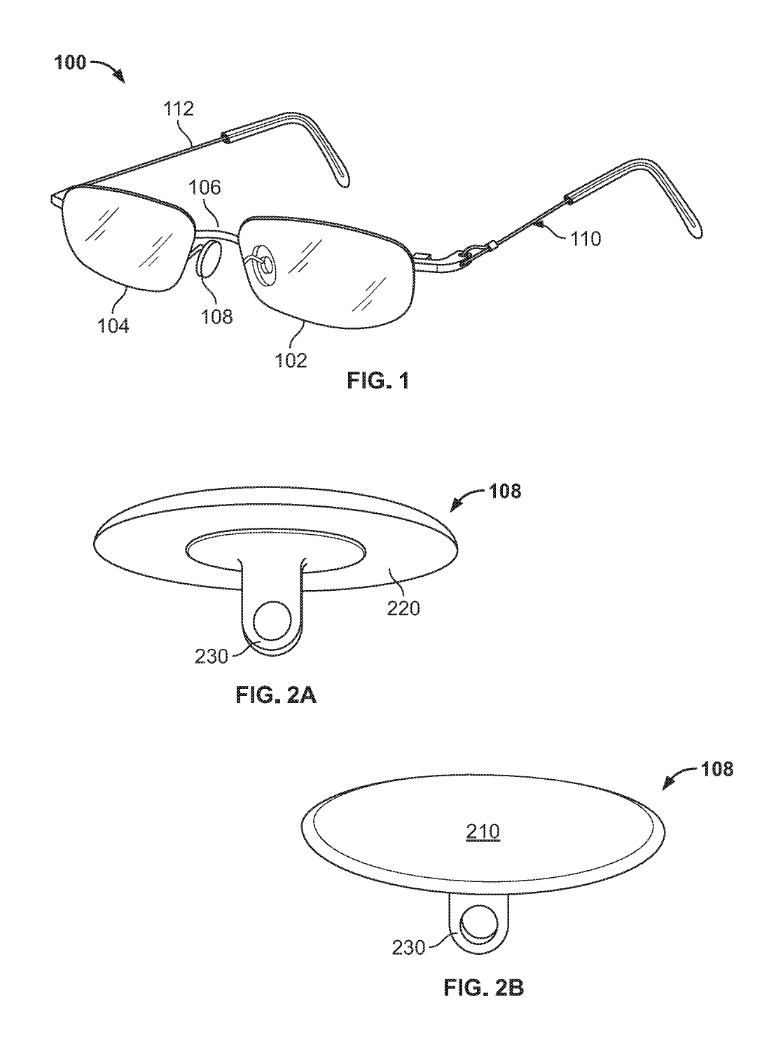

Reference will now be made in detail to exemplary embodiments of the present invention which are illustrated in the accompanying drawings. Wherever possible, the same reference numbers are used throughout the drawings to refer to the same parts. Some embodiments of the aromatherapy delivery system disclosed herein are depicted in FIGS. 1-8.

FIG. 1 depicts a perspective view of a pair of eyeglasses 100. The eyeglasses may comprise a right eye frame 102, a left eye frame 104, a bridge 106 connecting between the right eye frame and the left eye frame, a nose pad 108 attached to the right eye frame and to the left eye frame. The eyeglasses may further comprise a right temple 110 and a left temple 112 coupled to the right eye frame 102 and the left eye frame 104, respectively.

Nose pads 108 are one illustrative embodiment of nose support devices contemplated by the present invention. In some embodiments, the nose support device may comprise a nose pad, an eyeglass bridge, or a combination thereof. Nose pads disclosed herein may include, but not be limited to, one or more of slide-on nose pad, slide-in nose pad, screw-in nose pad, push-on nose pad, push-in nose pad, snap-on nose pad, snap-in nose pad, stick-on nose pad, adhesive nose pad. Eyeglass bridges disclosed herein may include, but not be limited to, a saddle bridge.

FIGS. 2A and 2B depict a blown up perspective view of one embodiment of nose pad 108. In certain embodiments, the nose support device may be a conventional nose pad similar to nose pad 108 illustrated in the figures. In certain embodiments, the nose support device may include a custom nose pad. For instance, the custom nose pad may be smaller than a conventional nose pad in a pair of eyeglasses. The nose support device may include a proximal side 210 and a distal side 220. The proximal side may be the side closer to the nose and the distal side may be the side farthest away from the nose. In some embodiments, the nose support device may further comprise an attachment mechanism 230 protruding from the distal side, designed to be affixed to an individual's eyeglasses. In other embodiments, the nose support device along with the eyeglasses may comprise a single component such that an additional attachment mechanism is absent or is an intact part of the eyeglasses. In yet other embodiments, the nose support device may be affixed to a pair of eyeglasses by an attachment mechanism without protrusions from the nose support device, such as through adhesion.

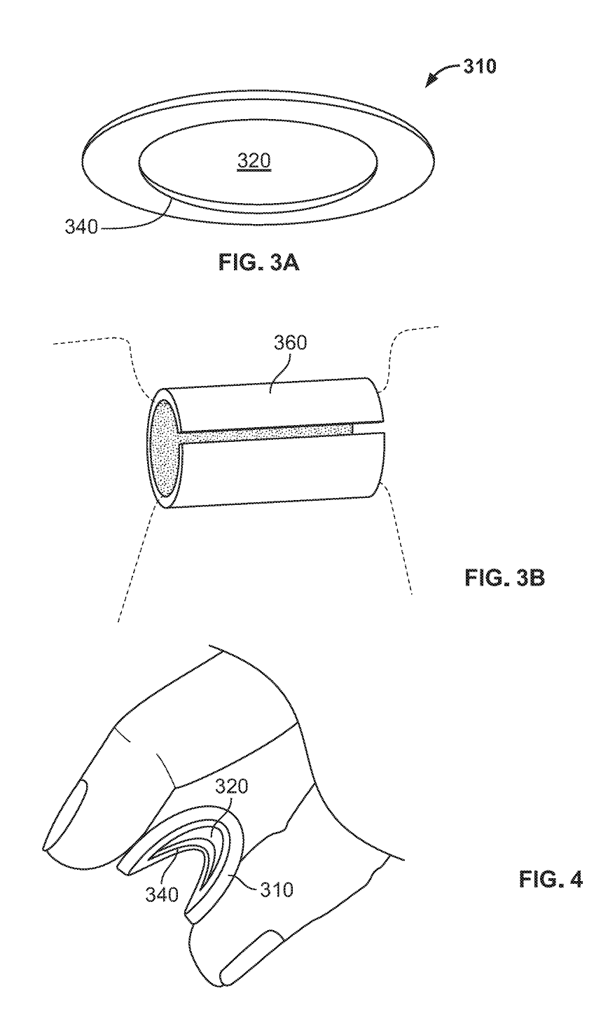

FIG. 3A depicts a perspective view of a cover for a nose support device according to an embodiment. The cover 310 may comprise an inner section 320. The inner section 320 may be configured to receive a medicinal product. The cover 310 may be also configured to receive a nose support device through an opening 340. In some embodiments, the cover along with the nose support device may be separate while in other embodiments they may be inseparable. The cover may be configured to receive the nose support device through an opening 340 such that the nose support device may be at least partially enclosed with the cover. In some embodiments, the nose support device may be fully enclosed with the cover. In other embodiments, the cover may coat the majority of the nose support device, while still leaving gaps near the opening of the cover. For example, if the nose support device is a nose pad 108, having an attachment mechanism 230 (such as an anchor), the cover 310 may coat the nose pad while leaving a gap around the anchor (see gaps 710 in FIG. 7).

FIG. 3B depicts a cover according to an embodiment of the invention. Cover 360 may be in a form of a sleeve configured to receive an eyeglass bridge similar to saddle bridge 106. The cover may comprise an inner section configured to receive a medicinal product.

FIG. 4 is a demonstration of the flexibility of cover 310 for a nose support device according to an embodiment. The cover may be flexible and the opening of the cover 340 may be elastic to accommodate nose support devices of various structures, configurations, and sizes. The cover may conform to any particular geometric dimension and may subscribe to various angular orientations to best accommodate a given nose support device.

The cover may comprise a material selected from the group consisting of plastic, metal, rubber, gel, air, paper or a combination thereof. In particular embodiments, the material of the cover may comprise at least one of silicone, nylon, vinyl, polyvinyl chloride, polycarbonate, titanium, latex, acetate, shape memory polymer materials, Air Active®, Bio-Feel®, air filled, gel filled, acrylic or combinations thereof. In one embodiment, the cover may be an overlying soft silicone cover that fits over a hard plastic eyeglass nose support device, such as a nosepad.

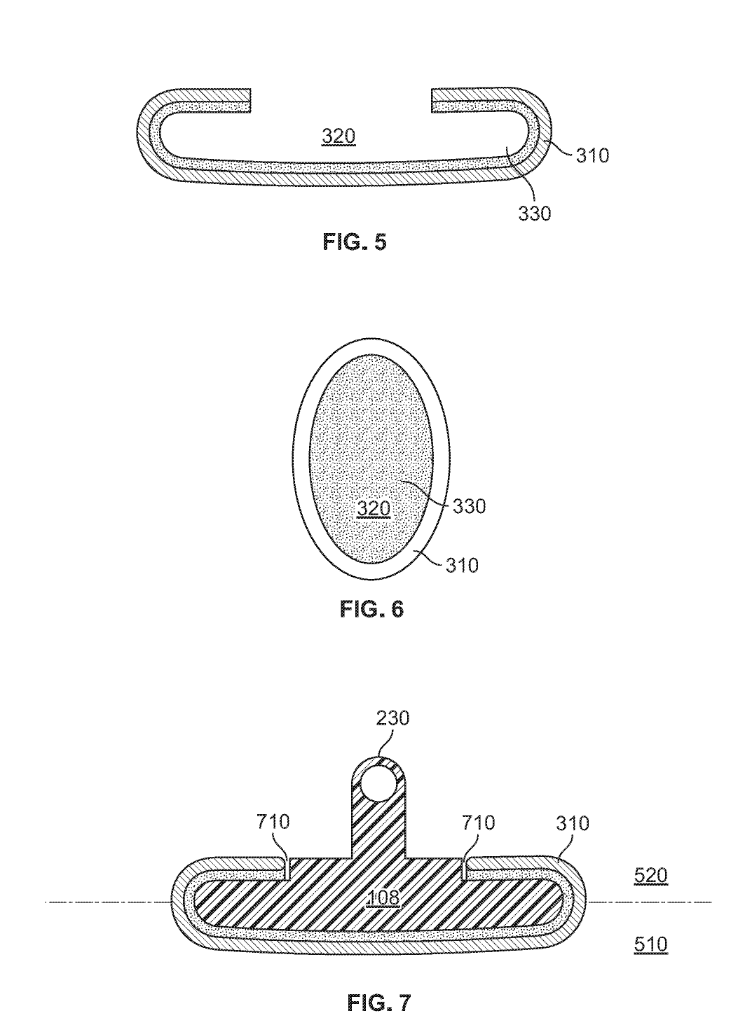

FIGS. 5 and 6 depict a side view and a top view, respectively, of cover 310 for a nose support device with medicinal product 330 without a nose support device. The cover 310 may be configured to receive a medicinal product 330. For instance, the inner section 320 of the cover may be lined with a medicinal product such as an aromatherapy depot. In some embodiments, the medicinal product may be an active agent selected from the group consisting of analgesics, anti-inflammatory agents, local and anti-asthmatic agents, anti-microbial agents, anti-migraine agents, anti-viral agents, cholinergic agonists, histamine antagonists, sedatives, prostaglandins, retinoids, aromatic oils, antibiotics, anti-irritant, glaucoma agents, essential oils, menthol, appetite suppressors, stimulants and mixtures thereof.

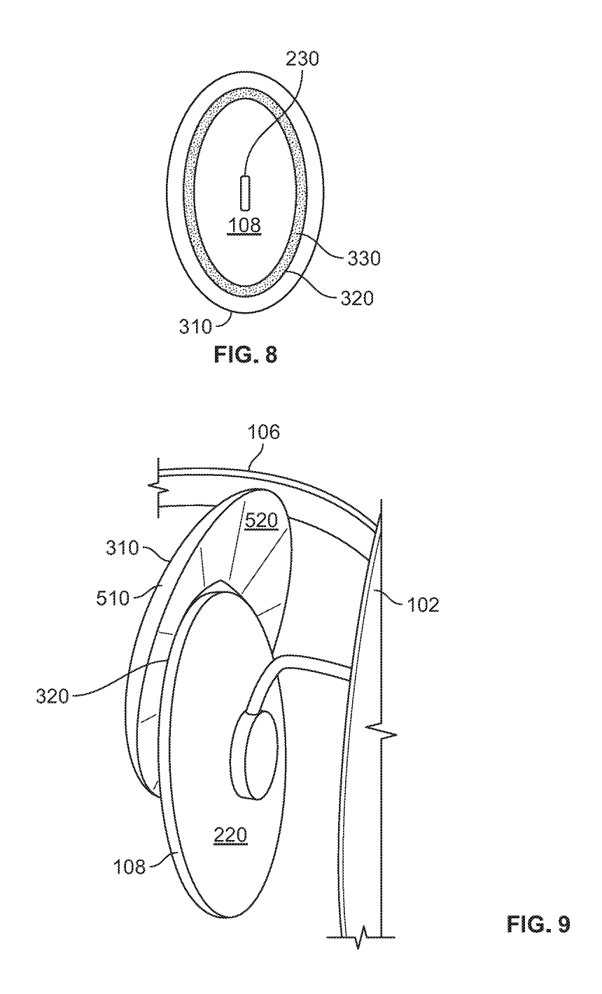

FIGS. 7 and 8 depict a side view and a top view, respectively, of a cover for a nose support device with medicinal product coated over a nose support device. The exemplary embodiment of FIGS. 7 and 8 illustrate an eye glass nose pad 108 that may be made of hard plastic. The nose pad is an illustrative nose support device acting as an anchor and providing structural support for an overlying soft cover 310. The overlying soft cover may have a medicinal product 330 coated on its inner lining located in opening 320.

In some embodiments, the cover may comprise a first section 510 and a second section 520. The first section 510 may be configured to be in direct contact with an individual's nose. The second section 520 may comprise any part of the cover not configured to be in direct contact with an individual's nose. In certain embodiments, the first section may be impermeable and the second section may be permeable such that the medicinal product may be emitted through the second section but not through the first section. In other embodiments, the first section may be permeable and the second section may be impermeable such that the medicinal product may be emitted through the first section, which is in direct contact with an individual's nose, but not through the second section.

In some embodiments, the cover may be impermeable in its entirety, such that the medicinal product lined in the cover may be emitted through gaps 710 formed near the opening of the cover. In certain embodiments, the cover may be permeable in its entirety, such that the medicinal product may be emitted through the cover 310 in addition to optionally being emitted through gaps 710 that may have been formed near the opening of the cover. In other embodiments, the cover may be semi-permeable such that various medicinal products could permeate through various sections of the cover. For instance, certain medicinal products could permeate through the cover whereas other medicinal products could only permeate through gaps formed near the opening of the cover.

In the embodiments disclosed herein, the medicinal product in the aromatherapy delivery system remains in close proximity to one's eyes and nose and may be used to treat an ailment by emitting aromatherapy to the individual's nose or eyes. The close proximity to one's eyes and nose result in aerial diffusion and/or direct inhalation of the medicinal product, thereby alleviating common human ailments.

The aromatherapy emission may occur continuously over a short or an extended period of time. For instance, in some embodiments, the period of time ranges from about 1 minute, about 5 minutes, about 10 minutes, about 15 minutes, about 30 minutes, about 45 minutes, about 1 hours, about 2 hours, about 3 hours, about 4 hours, or about 5 hours, to about 6 hours, about 7 hours, about 8 hours, about 9 hours, about 10 hours, about 11 hours, about 12 hours, about 24 hours, about 48 hours, or about 72 hours. In other embodiments, the period of time ranges from about 1 day, about 2 days, about 3 days, about 4 days, about 5 days, about 6 days, about 7 days, about 8 days, about 9 days, about 10 days, about 11 days, about 12 days, about 13 days, about 14 days, about 15 days to about 16 day, about 17 days, about 18 days, about 19 days, about 20 days, about 21 days, about 22 days, about 23 days, about 24 days, about 25 days, about 26 days, about 27 days, about 28 days, about 29 days, or about 30 days.

Covers according to embodiments of the invention may be interchangeable such that various covers may be lined with various medicinal products specific to an individual's targeted needs.

In certain embodiments, covers may have a long term use such that an individual could replenish the medicinal product lined in the inner section of the cover with a medicinal product cartridge. In some embodiments, it may be possible to attach the medicinal product cartridge directly to the inner section of the cover. In other embodiments, the cover may be lined with the medicinal product cartridge.

In certain embodiments, the cover may have a short term use and be disposable. For instance, it may be possible to obtain a plurality of disposable covers lined with the desired medicinal product and replace them hourly, daily, weekly, monthly, etc. It may also be possible to obtain a plurality of disposable covers that do not have the medicinal product lined in their inner section and to separately obtain various medicinal product cartridges that may be filled into the various covers.

For instance, an individual may purchase a eucalyptus essential oil and a lavender essential oil and use them interchangeably with the same long term cover or with a plurality of short term covers. Thus, the present invention is also directed to a method of forming aromatherapy delivery systems disclosed herein, comprising lining a cover for a nose support device with a medicinal product.

Common human ailments that may be alleviated by the continuous aromatherapy emission include, but are not limited to, at least one of ocular ailments, dry eyes, itchy eyes, burning eyes, eye ache, eye strain, tearing, foreign body sensation, headache, fatigue, eye fatigue, nasal congestion, lack of focus, visual impairment, eye disorder, glaucoma, macular degeneration, conjunctivitis, photophobia, red eye, blepharitis, keratitis, eye strain, blepharospasm, trachoma, and combinations thereof.

The aromatherapy delivery systems disclosed herein may be provided in a kit. The kit may include a cover configured to receive a nose support device through an opening and comprising an inner section configured to receive a medicinal product. In some embodiments, the kit may further comprise at least one of the following: a pair of eyeglasses, a nose support device, a medicinal product cartridge, and/or use and assembly instructions. In certain embodiments, the kit may comprise a cover that is already lined with a medicinal product, while in others a medicinal product may be absent from the cover.

In certain embodiments, the kit may comprise a pair of eyeglasses in which the nose support device is coated with the cover. Thus, the present invention is also directed to aromatherapy delivery systems, comprising a pair of eyeglasses with a nose support device and a cover such that the cover may be coated over the nose support device.

In other embodiments, the pair of eyeglasses may be separate from the cover. Thus, the present invention is also directed to a method of forming aromatherapy delivery systems disclosed herein, comprising slipping the cover over the nose support device as illustrated in FIG. 9.

In some embodiments, the aromatherapy delivery system may comprise a nose support device 108 and a cover 310 as two separate components. This allows an individual to slip the cover 310 over the nose support device 108 through the cover's opening 320. The nose support device may also be optionally attached to a pair of eyeglasses. In other embodiments, the aromatherapy delivery system may comprise a nose support device coated with a cover as a single component that may be attached to a pair of eyeglasses.

The nose support device provided in kit or as part of the aromatherapy delivery system may also have a long term or a short term use as previously described for the cover.

The aromatherapy delivery systems contemplated herein may be used with a variety of eyeglasses, including but not limited to, prescription glasses, sunglasses, fashion glasses, eyeglasses for recreational uses such as video games and/or movies, etc.

The individuals forming aromatherapy delivery systems disclosed herein may include, but not be limited to, manufacturers, opticians, consumers, etc. For instance, a consumer may select an appropriate medicinal product or aromatherapy depot. Then, an optician may line a cover with the chosen medicinal product. Lastly, a consumer may slip the cover containing the medicinal product onto the nose support device. Alternatively, the optician, consumer, or manufacturer may perform all of these steps, any number of these steps, and at any order.

### Examples

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

Study Exploring Whether Aromatherapy Emanating from an Individual's Eyeglass Nose Pad Alleviate Dry Eye Symptoms

The study explored whether aromatherapy through aerial diffusion in close proximity to an individual's eyes can alleviate dry eye symptoms. The effects of four different aromatherapy scents (eucalyptus, frankincense, lavender and peppermint) on four common dry eye symptoms (burning, tearing, foreign body sensation, and eye fatigue) were studied separately.

Procedure:

Selection criteria included 18 eyeglass wearing subjects who experienced dry symptoms at least several times daily. Each subject was asked to grade their baseline level of dry eye symptoms on a scale of 1-10. Subjects then applied a thin layer of aromatherapy oil to the inner portion of their nose pads. Subjects were then asked to re-grade their dry eye symptoms after 3 days of wearing eyeglasses with aromatherapy oil on their nose pads.

Results:

The percent of subjects who recorded improvements in their dry eye symptoms from wearing eyeglasses with aromatherapy infused nose pads is recorded in Table 1 below.

| | Symptoms | | | |
|---|---|---|---|---|
| | Reduced | | Reduced | |
| | Burning | Reduced | Foreign Body | Reduced Eye |
| Oil Type | Sensation | Tearing | Sensation | Fatigue |
| Peppermint | 80% | 75% | 80% | 88% |
| Eucalyptus | 68% | 80% | 75% | 80% |
| Lavender | 50% | 25% | 100% | 71% |
| Frankincense | 18% | 84% | 63% | 56% |

Results of this study showed varying degrees of symptomatic relief for each aromatherapy oil. Aerial diffusion of aromatherapy emanating from one's nose pad may be an important tool in alleviating dry eye symptoms.

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. An aromatherapy delivery system, comprising:
a cover configured to receive a nose support device through an opening, the cover comprising an inner section, the inner section configured to receive a medicinal product.

2. The aromatherapy delivery system of claim 1, wherein the inner section is lined with an aromatherapy depot.

3. The aromatherapy delivery system of claim 1, wherein the medicinal product is an active agent selected from the group consisting of analgesics, anti-inflammatory agents, local and anti-asthmatic agents, anti-microbial agents, anti-migraine agents, anti-viral agents, cholinergic agonists, histamine antagonists, sedatives, prostaglandins, retinoids, aromatic oils, antibiotics, anti-irritant, glaucoma agents, essential oils, menthol, appetite suppressors, stimulants and mixtures thereof.

4. The aromatherapy delivery system of claim 1, wherein the cover is impermeable in its entirety.

5. The aromatherapy delivery system of claim 1, wherein the cover is permeable in its entirety.

6. The aromatherapy delivery system of claim 1, wherein the cover comprises a first section configured to be in direct contact with an individual's nose, and a second section comprising any part of the cover not configured to be in direct contact with an individual's nose, wherein the first section is impermeable and the second section is permeable.

7. The aromatherapy delivery system of claim 1, wherein the nose support device comprises a nose pad, an eyeglasses bridge, or a combination thereof.

8. The aromatherapy delivery system of claim 7, wherein the nose pad comprises one or more of slide-on nose pad, slide-in nose pad, screw-in nose pad, push-on nose pad, push-in nose pad, snap-on nose pad, snap-in nose pad, stick-on nose pad, adhesive nose pad.

9. The aromatherapy delivery system of claim 7, wherein the eyeglass bridge comprises a saddle bridge.

10. The aromatherapy delivery system of claim 1, wherein the cover comprises a material selected from the group consisting of plastic, metal, rubber, gel, air, paper or a combination thereof.

11. The aromatherapy delivery system of claim 10, wherein the material comprises at least one of silicone, nylon, vinyl, polyvinyl chloride, polycarbonate, titanium, latex, acetate, shape memory polymer materials, Air Active, Bio-feel, Air Filled, Gel Filled, acrylic or combinations thereof.

12. The aromatherapy delivery system of claim 1, further comprising a medicinal product cartridge.

13. An aromatherapy delivery system, comprising:
a pair of eyeglasses, comprising:
a right eye frame;
a left eye frame;
a bridge connecting between the right eye frame and the left eye frame; and
a nose support device attached to the right eye frame, the left eye frame, the bridge, or a combination thereof,
a cover configured to receive the nose support device through an opening, the cover comprising an inner section, the inner section being configured to receive a medicinal product.

14. An aromatherapy delivery system of claim 13, wherein the cover is coated over the nose support device.

15. The aromatherapy delivery system of claim 13, wherein the inner section is lined with an aromatherapy depot.

16. The aromatherapy delivery system of claim 13, wherein the medicinal product is an active agent selected from the group consisting of analgesics, anti-inflammatory agents, local and anti-asthmatic agents, anti-microbial agents, anti-migraine agents, anti-viral agents, cholinergic agonists, histamine antagonists, sedatives, prostaglandins, retinoids, aromatic oils, antibiotics, anti-irritant, glaucoma agents, essential oils, menthol, appetite suppressors, stimulants and mixtures thereof.

17. The aromatherapy delivery system of claim 13, wherein the cover is impermeable in its entirety.

18. The aromatherapy delivery system of claim 13, wherein the cover is permeable in its entirety.

19. The aromatherapy delivery system of claim 13, wherein the cover comprises a first section configured to be in direct contact with an individual's nose, and a second section comprising any part of the cover not configured to be in direct contact with an individual's nose, wherein the first section is impermeable and the second section is permeable.

20. The aromatherapy delivery system of claim 13, wherein the nose support device comprises a nose pad, an eyeglasses bridge, or a combination thereof.

21. The aromatherapy delivery system of claim 20, wherein the nose pad comprises one or more of slide-on nose pad, slide-in nose pad, screw-in nose pad, push-on nose pad, push-in nose pad, snap-on nose pad, snap-in nose pad, stick-on nose pad, adhesive nose pad.

22. The aromatherapy delivery system of claim 20, wherein the eyeglass bridge comprises a saddle bridge.

23. The aromatherapy delivery system of claim 13, wherein the cover comprises a material selected from the group consisting of plastic, metal, rubber, gel, air, paper or a combination thereof.

24. The aromatherapy delivery system of claim 23, wherein the material comprises at least one of silicone, nylon, vinyl, polyvinyl chloride, polycarbonate, titanium, latex, acetate, shape memory polymer materials, Air Active, Bio-feel, Air Filled, Gel Filled, acrylic or combinations thereof.

25. The aromatherapy delivery system of claim 13, further comprising a medicinal product cartridge.

26.-77. (canceled)
